(19) United States
(12) Patent Application Publication
(10) Pub. No.: US 2016/0143877 A1
(43) Pub. Date: May 26, 2016

(54) COMPOSITION FOR SUPPRESSING ADIPOCYTE DIFFERENTIATION, FOR REDUCING FAT ACCUMULATION AND/OR FOR PROMOTING ADIPONECTIN SECRETION AND USAGE FOR SAID COMPOSITION

(71) Applicants: PUBLIC UNIVERSITY CORPORATION OSAKA CITY UNIVERSITY, Osaka-shi, Osaka (JP); KIYOMOTO IRON & MACHINERY WORKS CO LTD, Nobeoka-shi, Miyazaki (JP)

(72) Inventors: Akiko KOJIMA, Osaka-shi (JP); Isao YUASA, Osaka-shi (JP); Kunio KIYOMOTO, Nobeoka-shi (JP); Ayano OMURA, Nobeoka-shi, (JP)

(21) Appl. No.: 14/902,902

(22) PCT Filed: Jun. 2, 2014
(86) PCT No.: PCT/JP2014/064619

§ 371 Date: Jan. 5, 2016

(30) Foreign Application Priority Data
Jul. 5, 2013 (JP) 2013-142011

(51) Int. Cl. A61K 31/343

(52) U.S. Cl. CPC A61K 31/343

(57) ABSTRACT

The present invention relates to a composition for suppressing adipocyte differentiation, for reducing fat accumulation in adipocytes and/or for promoting adiponectin secretion from adipocytes, including sesaminol as an active component. The present invention also relates to a method for suppressing adipocyte differentiation, for reducing fat accumulation in adipocytes and/or for promoting adiponectin secretion of adipocytes, including administering a composition including sesaminol as an active component.

### TECHNICAL FIELD

The present invention relates to a composition for suppressing adipocyte differentiation, for reducing fat accumulation in adipocytes and/or for promoting adiponectin secretion of adipocytes, including sesaminol as an active component. The present invention also relates to a method for suppressing adipocyte differentiation, for reducing fat accumulation in adipocytes and/or for promoting adiponectin secretion of adipocytes, including administering the composition.

### BACKGROUND ART

With the change in lifestyle such as transfer of dietary habits towards Western style and lack of physical activity, the number of patients with dyslipidaemia, diabetes, hypertension, arteriosclerosis, metabolic syndrome is rapidly increasing. These diseases are also referred to as “lifestyle-related diseases” because lifestyle such as dietary habits and fitness habits affects pathogenesis and progression of the diseases. Lifestyle-related diseases are such severe diseases that they are involved in cardiac disease and cerebrovascular disease which are the main causes of death of Japanese.

Among lifestyle-related diseases, diabetes, hypertension, dyslipidaemia and arteriosclerosis are reported to be diseases to which obesity is involved as a risk factor. Therefore obesity is also regarded as a lifestyle-related disease. Prophylaxis, therapy and amelioration of obesity is suggested to be one of the important means for prophylaxis, therapy and amelioration of lifestyle-related diseases.

Obesity refers to an increase of body fat above the normal level. Fat is accumulated in adipocytes in adipose tissue in the body. In adults at ideal body weight, each adipocyte has a diameter of about 70 to 90 μm, while in obese adults the diameter of an adipocyte is enlarged to be about 130 μm. Thus, at the cell level, obesity refers to an enlargement of adipocytes due to accumulation of fat and an increase of the number of such adipocytes.

Adipocytes are differentiated from fibroblasts, which are preadipocytes. Preadipocytes cannot accumulate fat, and thus it is effective to suppress differentiation of preadipocytes into adipocyte in order to prevent and ameliorate obesity. Adipocytes having normal size secrete adiponectin which improves insulin resistance, while enlarged adipocytes have not only reduced secretion of adiponectin but also excess secretion of TNF-α, resistin and fatty acids which induce insulin resistance. Therefore it is also effective to reduce the amount of fat accumulated in adipocytes and reduce the size of adipocytes in order to prevent and ameliorate obesity.

Further adiponectin secreted from adipocytes as described above improves insulin resistance by activating AMPK (AMP-activated protein kinase) in skeletal muscle and liver to promote sugar uptake. Therefore, it is believed that adiponectin is effective for prophylaxis and amelioration of diabetes. Moreover adiponectin can suppress uptake of oxidized LDL by macrophages, and thus it is believed that adiponectin is also effective for prophylaxis and amelioration of atherosclerosis. Therefore promoting adiponectin secretion from adipocytes is effective for prophylaxis and amelioration of not only obesity but also diabetes and atherosclerosis.

There are various approaches for prophylaxis and therapy of lifestyle-related diseases including improvement in dietary habits and fitness habits. In recent years, compositions containing components effective for reduction of fats and suppression of adipocyte differentiation have been developed in order to prevent and ameliorate obesity which is a risk factor of various diseases. For example, Patent Literature 1 discloses an agent for reducing body fat containing sesamin, a type of lignans in sesame, and discloses that it was observed in rats received sesamin, the weight of adipose tissue around the kidney was reduced and triglyceride secretion from the liver was reduced.

### CITATION LIST

### Patent Literature

Patent Literature 1: Japanese Unexamined Patent Application Publication No. 2000-309533

### SUMMARY OF INVENTION

### Technical Problem

Although a number of components useful for prophylaxis and amelioration of lifestyle-related diseases has been discovered so far, there is still a need for a component having further excellent effects. With the foregoing in view, an object of the present invention is to provide a composition containing an active component that is more effective for prophylaxis, therapy and amelioration of lifestyle-related diseases.

### Solution to Problem

The inventors of the present invention found, as a result of extensive study, that sesaminol, a type of lignans in sesame, has abilities to suppress differentiation of preadipocytes into adipocytes, to reduce fat accumulation in adipocytes and to promote adiponectin secretion from adipocytes. Thereby the inventors have completed the present invention.

Thus the present invention provides a composition for suppressing adipocyte differentiation, for reducing fat accumulation in adipocytes and/or for promoting adiponectin secretion from adipocytes, including sesaminol as an active component.

The present invention also provides a method for suppressing adipocyte differentiation, for reducing fat accumulation in adipocytes and/or for promoting adiponectin secretion from adipocytes, including administering a composition containing sesaminol as an active component.

### Advantageous Effects of Invention

According to the present invention, differentiation of preadipocytes into adipocytes is suppressed, fat accumulation in adipocytes is reduced and secretion of adiponectin from adipocytes is promoted. Due to these actions, it is expected that lifestyle-related diseases are prevented, treated and/or ameliorated.

### BRIEF DESCRIPTION OF THE DRAWINGS

FIG. 1 is a photograph of Oil red 0 stained 3T3-L1 cells which was cultured with sesaminal during the differentiation period.

FIG. 2 is a graph showing an effect of sesaminol on glycerol-3-phosphate dehydrogenase (GPDH) activity in 3T3-L1 cells, which were cultured with sesaminol during the differentiation period. obtained by adding sesaminol during differentiation induction of 3T3-L1 cells;

FIG. 3 is a photograph of Oil red 0 stained 3T3-L1 cells which was cultured with sesaminal during the differentiation period;

FIG. 4 is a graph showing an effect of sesaminol on fat accumulation in 3T3-L1 adipocytes; and

FIG. 5 is a graph showing an effect of sesaminol on adiponectin secretion from 3T3-L1 adipocytes.

### DESCRIPTION OF EMBODIMENTS

A composition for suppressing adipocyte differentiation, for reducing fat accumulation in adipocytes and/or for promoting adiponectin secretion from adipocytes of the present invention (hereinafter also merely referred to as “composition”) includes sesaminol as an active component. Sesaminol is known as a lignan contained in sesame seeds and is a compound represented by the following structural formula:

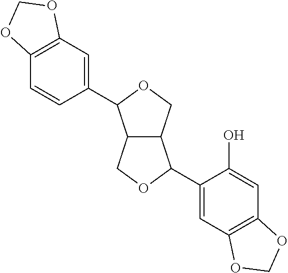 [SMILES: Oc1cc2c(cc1C1OCC3C(c4ccc5c(c4)OCO5)OCC13)OCO2]

Sesaminol used in the present invention may be derived from any source without limitation and may be sesaminol derived from plants such as sesame seeds or synthesised or semi-synthesised sesaminol. Methods per se for obtaining sesaminol from sesame seeds and the like are known and examples thereof include methods for producing sesaminol by the action of enzymes of certain microorganisms from raw materials including sesame seeds or defatted lees of the seeds (see Japanese Unexamined Patent Application Publication Nos. 2006-61115 and 2008-167712).

In the embodiments of the present invention, it is preferable to use sesaminol obtained from sesaminol glycoside in defatted lees of sesame seeds according to the method described in Japanese Unexamined Patent Application Publication No. 2008-167712. The obtained sesaminol may be directly used in the composition of the present invention or may be optionally subjected to processes such as concentration, dilution, filtration, deodorization, decolouration and drying.

In the embodiments of the present invention, the composition may only contain sesaminol; however the composition may appropriately contain other components which are generally used in the technical fields of quasi drug, pharmaceuticals, food or the like at an amount that does not deteriorate the action of sesaminol.

Examples of such other components include a binding agent (syrup, acacia gum, gelatine, sorbitol, tragacanth, polyvinylpyrrolidone, etc.), a filler (lactose, sucrose, corn starch, calcium phosphate, sorbitol, glycine, etc.), a lubricant (magnesium stearate, talc, polyethylene glycol, etc.), a disintegrating agent (starch, microcrystalline cellulose, etc.), a humectant (sodium lauryl sulphate, etc.), a suspending agent (sorbitol, syrup, methylcellulose, glucose syrup, gelatine, hydrogenated edible fat, etc.), an emulsifying agent (lecithin, sorbitan monooleate, acacia gum, etc.), a non-aqueous vehicle (almond oil, fractionated coconut oil or glycerol, propylene glycol, ethyl alcohol and the like hydrophobic esters, etc.), a preservative (methyl or propyl p-hydroxybenzoate, sorbic acid, etc.), a fragrance (synthetic fragrances, natural fragrances, etc.), a sweetener (sucrose, stevia, etc.), a pH-controlling agent (sodium hydrogen carbonate, potassium carbonate, etc.), powder (pigments, dyes, resins, etc.), a thickening agent (acacia gum, methylcellulose, etc.), an antioxidant (vitamin C, vitamin E, etc.) and the like.

The composition of the present invention may be administered by any method without limitation, and the method for administration may be appropriately selected from oral administration, injection (subcutaneous, intradermal, intramuscular, intravenous, intraarterial), administration on skin, transdermal administration and the like, among which oral administration or injection is preferred.

The dosage of the composition of the present invention is not particularly limited and may be appropriately selected according to the weight or health condition of the subject receiving the composition. For example, when the composition is orally administered to an adult, the dosage thereof may be 1 to 100 mg, preferably 1 to 50 mg and more preferably 2 to 10 mg as the amount of sesaminol per day. When the composition is administered by injection to an adult, the dosage may be 0.1 to 100 mg, preferably 0.1 to 50 mg and more preferably 0.2 to 10 mg as the amount of sesaminol per day. The frequency of administration of the composition of the present invention is not particularly limited and may be once or more daily.

The composition of the present invention may be provided as a composition for prophylaxis, therapy or amelioration of lifestyle diseases. Lifestyle diseases are defined as, in the art, a group of diseases in which lifestyle such as dietary habits, fitness habits, rest, smoking or drinking is involved in development and progression of the diseases. The composition of the present invention is particularly suitable for diseases or conditions which develop and progress due to dietary habits and/or fitness habits. Examples of such diseases and conditions include obesity, dyslipidaemia (particularly hypertriglyceridemia), diabetes (particularly type II diabetes), hypertension, arteriosclerosis (particularly atherosclerosis), metabolic syndrome and the like. Metabolic syndrome as used herein means the condition in which visceral fat accumulation is combined with two or more of hyperglycaemia, hypertension and dyslipidaemia.

The composition of the present invention may be administered to mammalians including humans. Particularly, healthy subjects and patients with lifestyle-related diseases are suitable. The composition of the present invention may be administered to healthy subjects for prophylaxis of lifestyle-related diseases and to patients with lifestyle-related diseases for therapy or amelioration of the diseases.

The composition of the present invention may be in any form of a quasi drug, pharmaceutical drug, food or research reagent, as far as sesaminol can be administered, among which a quasi drug, pharmaceutical drug or food is preferable.

More specifically, examples of quasi drug or pharmaceutical drug include tablets, pills, powders, granules, capsules, liquids and solutions, suspensions, emulsions, injections, drops and the like. Examples of food include beverages (nutritious supplement drinks), food (including Food with Nutrient Function Claims, Food for Specified Health Uses), supplements (tablets, capsules, granules, etc.), food for patients (food prepared for hospital feeding, food prepared for care receivers, etc.) and the like. The composition of the present invention may alternatively be in the form of food additive (in the form of liquid, powder, paste, etc.) or may be added to existing seasonings or the like as a food additive. The composition in the forms described above may be produced according to well-known methods in the art.

The composition of the present invention may contain any amount of sesaminol without limitation. The amount of sesaminol may be appropriately selected according to the form of the composition. For example, the amount of sesaminol relative to the total weight of the composition may be 0.0001 to 50% by weight, preferably 0.0005 to 40% by weight and more preferably 0.001 to 35% by weight.

The scope of the present invention also encompasses use of sesaminol for manufacturing a composition for suppressing adipocyte differentiation, for reducing fat accumulation in adipocytes and/or for promoting adiponectin secretion from adipocytes. The composition and sesaminol are as described herein above.

The scope of the present invention also encompasses a method for suppressing adipocyte differentiation, for reducing fat accumulation in adipocytes and/or for promoting adiponectin secretion of adipocytes, including administering a composition containing sesaminol as an active component. Sesaminol and the composition containing sesaminol as an active component are as described hereinabove. The method for administration and dosage of the composition are also as described hereinabove.

The scope of the present invention further encompasses use of sesaminol for suppressing adipocyte differentiation, for reducing fat accumulation in adipocytes and/or for promoting adiponectin secretion from adipocytes. Sesaminol is as described hereinabove.

The present invention is hereinafter more specifically described by way of Examples which do not limit the present invention.

### EXAMPLES

### Preparation Example

### Preparation of Sesaminol

In this Example the sesaminol used was obtained from sesaminol glycoside contained in sesame defatted lees by culturing Paenibacillus sp. KB0549 strain (Accession No.: FERM P-21057) in a medium containing sesame defatted lees according to the method described in Japanese Unexamined Patent Application Publication No. 2008-167712. Specifically, sesaminol was prepared as follows.

KB0549 strain was grown in a medium containing a warm water extract of sesame defatted lees (available from Takemoto Oil & Fat Co., Ltd.), 1.0% tryptone, 0.5% yeast extract and 0.89% NaCl to obtain a KB0549 culture solution. The obtained culture solution was added to sesame defatted lees (10.0 kg; heat sterilised and adjusted to water content of 70% and pH 6.0) and subjected to fermentation in a solid fermenter at 37° C. while continuing intermittent stirring and aeration over 6 days.

The fermented sesame defatted lees were dried to have a water content of 8.5%. To the dried material, 95% ethanol was added at a proportion of 100 L per 10.0 kg of the dried material and heated to 50° C. while stirring to extract sesaminol. The resulting liquid extract was subjected to diatomaceous earth filtration by filter pressing to remove solid matters and obtain a liquid filtrate (82 L). The resulting filtrate was concentrated to 4.1 L in a vacuum concentrating apparatus. To the resulting concentrated solution, 4 times or more volume of 99.5% ethanol were added and insoluble matters were removed by filtration through a filter paper. The obtained solution was concentrated on an evaporator to give a highly concentrated sesaminol solution (4.05 L).

The resulting highly concentrated solution was analysed on a high performance liquid chromatography (HPLC) in order to identify sesaminol and sesaminol-related compounds contained therein. As a result, it was found that the concentrated solution (4.05 L) contained 18.4 g of sesaminol. HPLC analysis was carried out under the following conditions:

### HPLC: HITACHI LaChrom

### Column: Wakosil-II 5C18HG (φ 4.6*250 mm, Wako Pure Chemical Industries, Ltd.)

Development solvent: A: 10% acetonitrile+0.1% trifluoroacetic acid, B: 80% acetonitrile+0.1% trifluoroacetic acid, a linear gradient of B from 10% to 100% in 40 minutes.
Flow rate: 0.8 ml/min
Analysis wavelength: 280 nm

The highly concentrated sesaminol solution was dissolved in dimethyl sulphoxide (DMSO) to prepare a sesaminol solution (3.0 mg/ml) to be used in Examples. The inventors of the present invention confirmed that the resulting sesaminol solution was not toxic to and did not affect viability of various cell lines used in Examples.

### Example 1

### Effect of Sesaminol on Adipocyte Differentiation

### (1) Investigation of Triacylglycerol (TG) Accumulation

Preadipocytes, when differentiated into adipocytes, intracellularly accumulate lipid droplets containing TG and the like. In the present Example, sesaminol was added to preadipocyte during induction of differentiation and intracellular TG after completion of induction was stained with an azo dye, Oil red 0, to evaluate an effect of sesaminol on adipocyte differentiation.

(1-1) Cell Culture

Mouse fibroblast cell line 3T3-L1 (Japan Health Sciences Foundation) were used as preadipocytes. 3T3-L1 cells were inoculated at a concentration of 1.0×10⁵ to 1.0×10⁶ cells/ml and cultured until they reached 80 to 90% confluence in a 5% CO₂ incubator at 37° C. The culture medium used was Dulbecco's modified Eagle's medium (DMEM: Nissui Pharmaceutical Co., Ltd.) containing 10% fetal bovine serum (FBS: Nichirei Biosciences, Inc.), penicillin (50 units/ml, Meiji Co., Ltd.) and streptomycin (50 mg/ml, Meiji Co., Ltd.).

(1-2) Induction of Differentiation of Preadipocytes into Adipocytes

After culturing the cells until they reached 80 to 90% confluence, the medium was replaced with DMEM containing differentiation inducing agents which were bovine insulin (0.2 μM, Wako Pure Chemical Industries, Ltd.), dexamethasone (0.25 μM, Wako Pure Chemical Industries, Ltd.) and 3-isobutyl-1-methylxanthine (0.5 mM, Wako Pure Chemical Industries, Ltd.) (this is referred to as “Day 0 of differentiation induction”). After two days, the medium was replaced with DMEM containing only insulin (0.2 μM). Thereafter, the medium was replaced with fresh DMEM every two days and induction of differentiation was completed on Day 8 of differentiation induction. The sesaminol solution (final concentration of sesaminol: 0.5 or 1.0 μg/ml) was added at the time of replacement of medium on Days 0, 2, 4 and 6 of differentiation induction. As a control, cells were differentiated without addition of the sesaminol solution.

(1-3) Evaluation of Adipocyte Differentiation Based on TG Accumulation

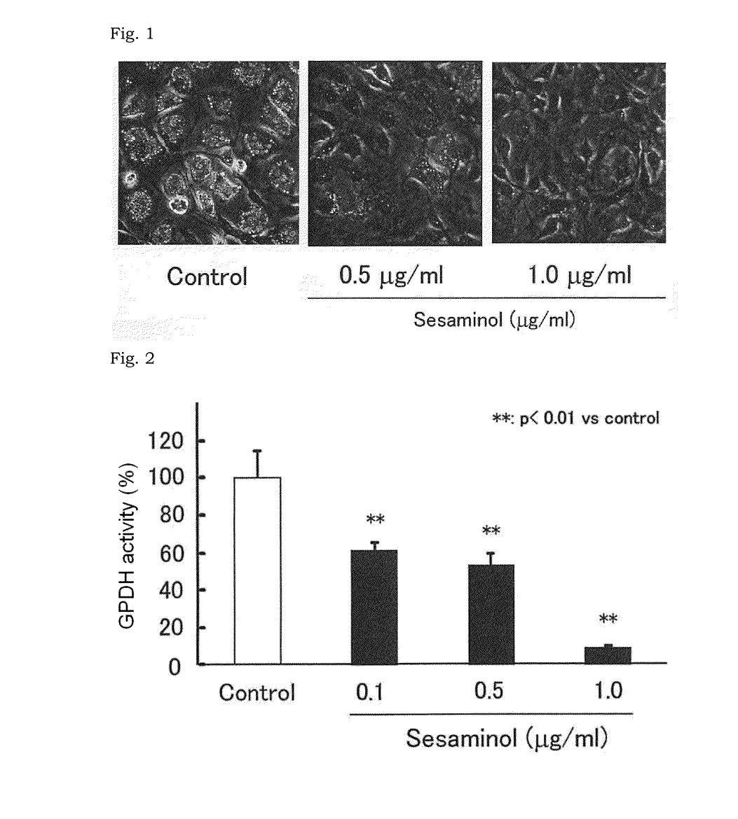

An Oil red 0 staining solution used in the present Example was an Oil red 0/isopropanol saturated solution obtained by dissolving Oil red 0 (Wako Pure Chemical Industries, Ltd.) in isopropanol (Wako Pure Chemical Industries, Ltd.). The Oil red 0 staining solution and ultrapure water were mixed at a volume ratio of 6:4 and after 10 minutes, the mixed solution was filtered through a 0.45 μm filter. Cells after differentiation induction were separated from the medium and washed twice with phosphate buffered saline (PBS). Cells were fixed with 70% ethanol for 30 seconds and stained with the Oil red 0 staining solution (2 ml) at room temperature for 2 hours. The stained cells were washed with 50% ethanol for 10 seconds and further washed with deionised water until water did not turn red any more. After washing, cells were analysed under a microscope (OLYMPUS IX-70: Olympus Corporation) and photographed. FIG. 1 shows photograph of the samples.

From FIG. 1, it was found that almost all the control cells were stained for lipid droplets. This indicates that differentiation of preadipocytes into adipocytes was properly induced. Meanwhile, it was found that many cells in the medium to which sesaminol was added to the final concentration of 0.5 μg/ml during differentiation induction did not contain stained lipid droplets. Moreover, almost all the cells in the medium to which sesaminol was added to the final concentration of 1.0 μg/ml did not contain stained lipid droplets. These results suggest that differentiation of preadipocytes into adipocytes was suppressed when sesaminol was added during differentiation induction. Therefore, it is demonstrated that sesaminol has the ability to suppress differentiation of preadipocytes into adipocytes.

### (2) Investigation of Activity of Glycerol-3-Phosphate Dehydrogenase (GPDH)

GPDH is a rate-limiting enzyme of triacylglycerol (TG; also referred to as triglyceride) synthesis in adipocytes. Because the activity of GPDH is drastically enhanced when preadipocytes are differentiated into adipocytes, GPDH is also known as a marker of adipocyte differentiation. Thus GPDH activity of cells after differentiation induction was measured according to the method of Wise and Green (Wise, L. S. and Green, H., (1979) Participation of one isozyme of cytosolic glycerophosphate dehydrogenase in the adipose conversion of 3T3 cells. The Journal of Biological Chemistry, 254, 273-275). Cells used for the GPDH assay were obtained in the same manner as described in (1-1) and (1-2). As a control, cells were differentiated without addition of the sesaminol solution.

(2-1) Preparation of Samples and Measurement of Activity

Adipocytes derived from 3T3-L1 cells on or after Day 8 of induction were washed twice with PBS. Cells were added with a buffer for enzymatic extraction (100 mM triethanolamine, 2.5 mM EDTA) (300 μl) and collected using a cell scraper. The collected cells were disrupted by ultrasonic (middle, 2.5 minutes, 10 seconds/20 seconds) and immediately centrifuged (13,000 rpm, 5 minutes, 4° C.) to obtain a supernatant. The resulting supernatant was used as a measurement sample.

(2-2) Measurement of GPDH Activity

To a test tube containing the buffer for enzymatic extraction (2800 μl) which was maintained at 28° C. in a water bath, 2-mercaptoethanol (final concentration: 0.1 mM), NADH (final concentration: 0.12 mM), dihydroxyacetone phosphate (DHAP: SIGMA) (final concentration: 0.2 mM) and a measurement sample (50 μl) were added and the absorbance was measured over 3 minutes at 340 nm. GPDH activity was calculated using an extinction coefficient of NADH (6.22 mM⁻¹cm⁻¹) on the basis of the change of absorbance per minute (NADH variation). The results are shown in FIG. 2. In FIG. 2, the enzyme activity of measurement samples is represented as a percent ratio with the enzyme activity of the control sample being 100%. In FIG. 2, ** denotes p<0.01 in relation to the control.

From FIG. 2, it was found that GPDH activity of cells after differentiation induction decreased with an increase in the amount of sesaminol added. This result suggests that differentiation into adipocytes was suppressed in preadipocytes to which sesaminol was added during differentiation induction. Therefore, it is demonstrated that sesaminol has the ability to suppress differentiation of preadipocytes into adipocytes.

### Example 2

### Effect of Sesaminol on Fat Accumulation in Adipocytes

### (1) Preparation of Adipocytes by Differentiation Induction

In the same manner as in (1-1) in Example 1, preadipocyte 3T3-L1 cells were cultured until they reached 80 to 90% confluence. Then the medium was replaced with DMEM containing differentiation inducing agents which were bovine insulin (0.2 μM, Wako Pure Chemical Industries, Ltd.), dexamethasone (0.25 μM, Wako Pure Chemical Industries, Ltd.) and 3-isobutyl-1-methylxanthine (0.5 mM, Wako Pure Chemical Industries, Ltd.) (this is referred to as “Day 0 of differentiation induction”). After two days, the medium was replaced with DMEM containing only insulin (0.2 μM). Thereafter, the medium was replaced with fresh DMEM every two days and induction of differentiation was completed on Day 8 of differentiation induction to obtain adipocytes.

### (2) Evaluation of TG Accumulation

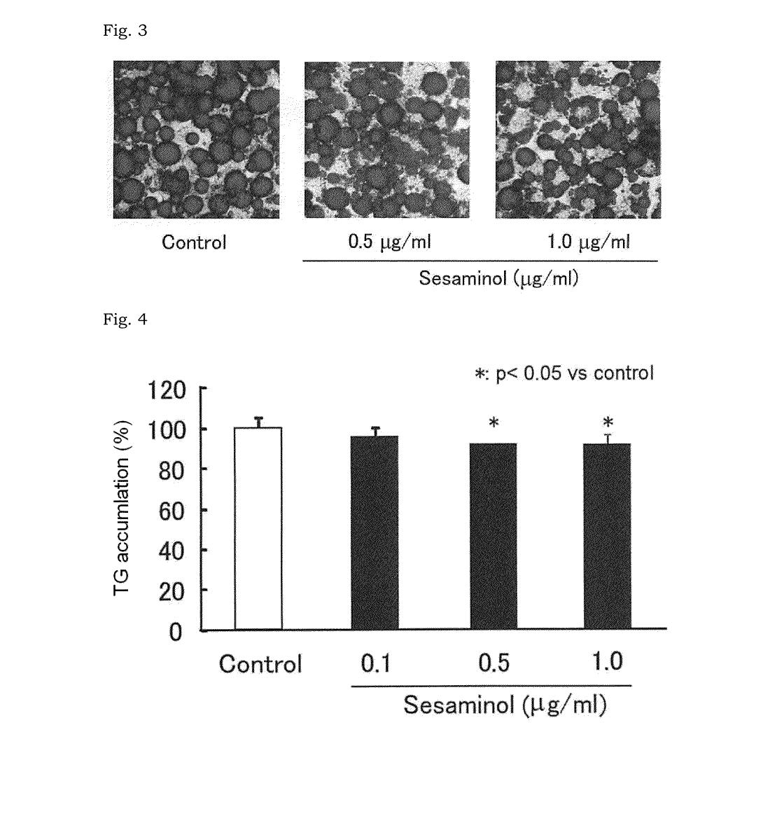

To the obtained adipocytes, the sesaminol solution was added so as to obtain the final concentration of sesaminol of 0.5 or 1.0 μg/ml. As a control, cells were cultured without addition of the sesaminol solution. After three days, cells were stained with the Oil red 0 staining solution and washed in the same manner as in (1-3) in Example 1. Cells were analysed under a microscope (OLYMPUS IX-70: Olympus Corporation) and photographed. FIG. 3 shows photographic images of the samples. After microscopic observation, cells were added with isopropanol (3 ml) to extract Oil red 0 staining solution and the resulting extract was measured for absorbance at 520 nm. According to the measured values, TG accumulations in cells were compared. The results are shown in FIG. 4. In FIG. 4, the TG accumulation of samples is represented as a percent ratio with the accumulation in the control being 100%. In FIG. 4, * denotes p<0.05 in relation to the control.

From FIG. 3, it was found that lipid droplets in adipocytes treated with sesaminol were tend to decrease the size compared to the control. From FIG. 4, it was found that addition of sesaminol decreased TG accumulation in adipocytes. These results demonstrate that sesaminol has the ability to reduce fat accumulation in adipocytes.

### Example 3

### Effect of Sesaminol on Adiponectin Secretion from Adipocytes

Various physiologically active adipocytokines are secreted from adipocytes. Among these adipocytokines, adiponectin is known to improve insulin resistance and prevent atherosclerosis. In the present Example, the profile of adiponectin secretion after addition of sesaminol to adipocytes was analysed by SDS-PAGE and western blotting.

(1) Preparation of Adipocytes by Differentiation Induction

In the same manner as in (1-1) in Example 1, preadipocyte 3T3-L1 cells were cultured until they reached 80 to 90% confluence. Then the medium was replaced with DMEM containing differentiation inducing agents which were bovine insulin (final concentration: 0.2 μM, Wako Pure Chemical Industries, Ltd.), dexamethasone (final concentration: 0.25 μM, Wako Pure Chemical Industries, Ltd.) and 3-isobutyl-1-methylxanthine (final concentration: 0.5 mM, Wako Pure Chemical Industries, Ltd.) (this is referred to as “Day 0 of differentiation induction”). After two days, the medium was replaced with DMEM containing only insulin (final concentration: 0.2 μM). Thereafter, the medium was replaced with fresh DMEM every two days and induction of differentiation was completed on Day 8 of differentiation induction to obtain adipocytes.

(2) Evaluation of Adiponectin Expression Level

The sesaminol solution was added so as to obtain the final concentration of sesaminol of 0.5 or 1.0 μg/ml in adipocytes. As a control, cells were cultured without addition of the sesaminol solution. After three days, cells were washed once with PBS and then collected using a cell scraper into HEPES buffer in a 1.5-ml tube. The tube was centrifuged (10000 rpm, 10 seconds, 4° C.) to remove the supernatant and collect the precipitate (cells). To the collected cells, an appropriate amount of lysis buffer (1% NP-40 and 1% Triton®-X100, 1 mM PMSF, 10 μg/ml leupeptin and 10 μg/ml aprotinin) was added and the tube was voltexed. The tube was cooled on ice for 30 minutes followed by ultrasonic treatment (2 cycles of 5-minute treatment and 30-second interval) to disrupt cells. The tube was centrifuged (15000 rpm, 4° C., 10 minutes) and a supernatant containing soluble cell lysate was collected. An aliquot of the collected supernatant was subjected to protein quantification using Serva Blue G (Serva Electrophoresis GmbH) based on the absorbance at 595 nm in order to calculate the amount of sample applied to electrophoresis.

Equal volumes of the supernatant containing cell lysate and a sample buffer were mixed to prepare a sample for electrophoresis. The obtained sample was subjected to SDS-PAGE electrophoresis and transfer onto a PVDF membrane according to standard methods. The membrane after transfer was blocked with 5% skimmed milk and then subjected to reaction with the primary antibody indicated below followed by washing in PBS for 5 minutes. The membrane after reaction with the primary antibody was then subjected to reaction with the secondary antibody indicated below and washed in PBS for 5 minutes. The membrane was then subjected to reaction in a DAB solution (3,3′-diaminobenzidine tetrahydrochloride (10 mg), distilled water (1 ml), 0.2 M PBS (50 ml) and 30% H₂O₂ (100 μl)), dried and then subjected to quantification of intensity of bands using Scion Image (free software: Scion). The ratio of the band intensity of adiponectin relative to the band intensity of β-actin was calculated for the samples.

Primary and secondary antibodies used in the present Example are as follows:

### (Primary Antibodies)

Rabbit anti-adiponectin antibody (Theromo SCIENTIFIC)

Mouse anti-actin antibody (ACTNO5 (c4)) (Abeam)

### (Secondary Antibodies)

Biotin-conjugated anti-rabbit IgG antibody (Dako A/S)

Biotin-conjugated anti-mouse IgG antibody (Dako A/S)

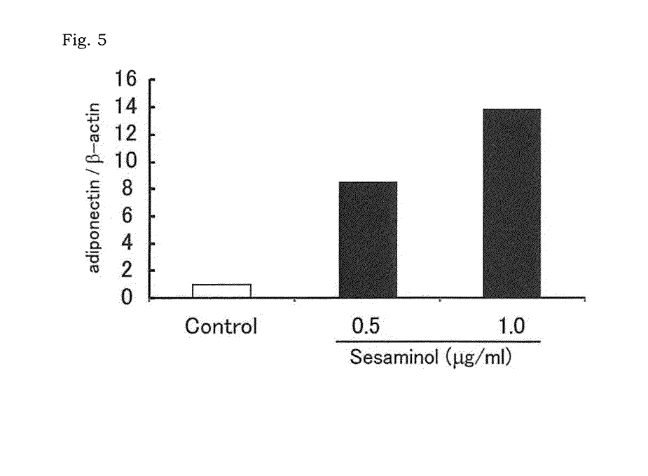

The results are shown in FIG. 5. In FIG. 5, the ratio in cells to which sesaminol was added is represented with the ratio in control cells being 1. From FIG. 5, it is found that adiponectin expression level was significantly enhanced with an increase in the amount of sesaminol added. Therefore, it is demonstrated that sesaminol has the ability to promote adiponectin secretion from adipocytes.

### Formulation Examples

Formulation Examples of the composition of the present invention are described hereinbelow. Sesaminol used is the one obtained by the method described in Preparation Example as above. In the following Formulation Examples, the amounts of components are denoted by “% by weight”.

### Formulation Example 1

### Cream

A cream is prepared as follows. Components indicated in (A) are mixed and the mixture is heated at 80° C. Separately, components indicated in (B) are mixed and the mixture is heated at 80° C. To the mixture of (A), the mixture of (B) is gradually added while stirring to emulsify followed by cooling to 35° C. to give a cream.

| | |
|---|---|
| (A) | |
| Squalene | 15.00 (% by weight) |
| Octyldodecyl myristate | 4.00 |
| Hydrogenated soybean phospholipid | 0.20 |
| Butyl alcohol | 2.40 |
| Hydrogenated oil | 1.50 |
| Stearic acid | 1.50 |
| Lipophilic glyceryl monostearate | 1.50 |
| Polyglyceryl monostearate | 0.50 |
| Behenyl alcohol | 0.80 |
| Polyglyceryl monomyristate | 0.70 |
| Bleached beeswax | 0.30 |
| d-δ-Tocopherol | 0.05 |
| Methylparaben | 0.30 |
| Sesaminol | 1.00 |
| (B) | |
| C10-30 alkyl-modified carboxyvinyl polymer | 0.20 |
| Carboxyvinyl polymer | 0.10 |
| 1,3-Butanediol | 18.00 |
| Sodium hydroxide | 0.10 |
| Purified water | 51.85 |
| | (Total: 100.00) |

### Formulation Example 2

### Skin Toner

A skin toner is prepared as follows. Components indicated in (A) are mixed and the mixture is heated at 80° C. to mix and homogenise followed by cooling. Components indicated in (B) are then sequentially added at 35° C. to mix and homogenise in order to obtain a skin toner.

| | |
|---|---|
| (A) | |
| Squalene | 0.40 (% by weight) |
| Polyglyceryl monolaurate | 0.10 |
| Long chain α-hydroxy fatty acid | 0.01 |
| Glycerol | 3.00 |
| 1,3-Butylene glycol | 8.00 |
| (B) | |
| Phenoxyethanol | 0.20 |
| 1,3-Butylene glycol | 10.00 |
| Allantoin | 0.10 |
| Citric acid | 0.01 |
| Sodium diethylenetriaminepentaacetate solution | 0.05 |
| Succinyl collagen solution | 0.01 |
| Purified water | 77.12 |
| | (Total: 100.00) |

Tablets

Tablets are prepared as follows. The following components are mixed and homogenised followed by moulding on a tableting machine to obtain tablets.

| | |
|---|---|
| Sesaminol | 5.00 (% by weight) |
| DL-α-Tocophorol acetate | 1.33 |
| Silicic acid anhydride | 1.50 |
| Calcium stearate | 1.33 |
| Lactose | 45.67 |
| Hydroxypropylcellulose | 2.67 |
| Microcrystalline cellulose | Balance |
| | (Total: 100.00) |

The present application relates to Japanese Patent Application No. 2013-142011 filed on 5 Jul. 2013, of which Claims, Specification, Drawings and Abstract are entirely incorporated herein by reference.

1-8. (canceled)

9. A method for suppressing adipocyte differentiation, for reducing fat accumulation in adipocytes and/or for promoting adiponectin secretion from adipocytes, comprising administering a composition including sesaminol as an active component.

10. The method according to claim 9, wherein the composition is for prophylaxis, therapy or amelioration of lifestyle-related disease.

11. The method according to claim 10, wherein the lifestyle-related disease is a disease or symptom that develops and progresses due to dietary habits and/or fitness habits

12. The method according to claim 10 or 11, wherein the lifestyle-related disease is obesity, dyslipidaemia, diabetes, hypertension, atherosclerosis or metabolic syndrome.

13-15. (canceled)
